(19) United States
(12) Patent Application Publication
(10) Pub. No.: US 2025/0177612 A1
(43) Pub. Date: Jun. 5, 2025

(54) BIODEGRADABLE IMPLANT AND METHOD OF DETERMINING A DEGRADATION TIME OF A BIORESORBABLE IMPLANT

(71) Applicant: MgSANA Corp., Mobile, AL (US)

(72) Inventor: Amir ELIEZER, Omer (IL)

(21) Appl. No.: 18/840,468

(22) PCT Filed: Feb. 24, 2023
(86) PCT No.: PCT/IB2023/000031

§ 371 Date: Aug. 21, 2024

(30) Foreign Application Priority Data
Feb. 26, 2022 (DE) 10 2022 000 698.7

(51) Int. Cl. A61L 27/58; A61L 27/32

(52) U.S. Cl. CPC A61L 27/58; A61L 27/32

(57) ABSTRACT

The disclosure relates to a biodegradable implant, comprising magnesium or a magnesium alloy, which is covered with a calcium phosphate, in particular hydroxyapatite, coating, wherein the implant has an impedance measured with electrochemical impedance spectroscopy (100 kHz-0.1 Hz) of Zᵢₘ>10,000Ω, preferably >15,000Ω, in particular 10,000-60,000Ω, preferably 25,000-60,000Ω, and/or Zᵣₑ>50,000Ω, preferably >100,000Ω, in particular 50,000-300,000Ω, preferably 250,000-300,000Ω.

### CROSS-REFERENCE TO RELATED APPLICATIONS

The present application is the national stage entry under 35 U.S.C. § 371 of International Patent Application No. PCT/IB2023/000031, which was filed Feb. 24, 2023, and claims the benefit of German Patent Application No. 10 2022 000 698.7, which was filed Feb. 26, 2022.

### FIELD OF THE DISCLOSURE

The present disclosure relates to a biodegradable implant and to a method of determining a degradation time of a bioresorbable implant. In particular, the present disclosure relates to magnesium implants which are coated with a calcium phosphate layer. In further, the present disclosure relates to a bioreactor, which is used for determining the degradation rate of a bioresorbable implant.

### BACKGROUND

It is well-known that magnesium degrades in the human body. Also with respect to the mechanical properties, magnesium and magnesium alloys are suitable to provide bioresorbable implants, e.g. bone plates and screws.

However, pure magnesium has a high corrosion rate, resulting in undesired quick weakening of the mechanical properties and undesired gas formation.

The corrosion rate can be decreased by using specific magnesium alloys, in particular Yttrium containing alloys. However, this is also not sufficient for many applications and my result in releasing harmful substances.

Document WO 2019/002277 A1 shows a calcium, manganese and zinc containing magnesium alloy which is coated with a hydroxyapatite layer.

The hydroxyapatite layer provides an improved biocompatibility of the implant and protects the magnesium alloy in the initial phase after implantation.

### SUMMARY

Given this background, it is an object of the present disclosure to provide an implant with improved corrosion properties and/or improved biocompatibility.

It is a further object of the present disclosure to provide a reliable non-destructive method for predicting the in-vivo corrosion properties of a coated implant.

The object of the present disclosure is archived by a biodegradable implant, by a method for determining a degradation time of a biodegradable implant and by a bioreactor according to the subject matter of the independent claims.

Specific examples and refinements of the present disclosure are defined in the dependent claims, the description and the drawings.

The present disclosure relates to a biodegradable implant, comprising magnesium or a magnesium alloy, which is covered with a calcium phosphate coating.

Preferably, the coating is embodied as a hydroxyapatite coating.

According to the present disclosure, the implant has an impedance measured with electrochemical impedance spectroscopy (100 kHz-0.1 Hz) of Zᵢₘ>10,000Ω, preferably >15,000Ω, in particular 10,000-60,000Ω, preferably 25,000-60,000Ω, and/or Zᵣₑ>50,000Ω, preferably >100,000Ω, in particular 50,000-300,000Ω, preferably 250,000-300,000Ω.

The inventor discovered that such a high impedance, in particular such a high Zᵣₑ results in an artificial bone-like surface with an increased resistance against corrosion.

It is to suspect that the impedance correlates with the applied energy for applying the calcium phosphate layers.

High energy results in a more covalent bonding which increases the corrosion resistance of the layer and also results in an increased biocompatibility.

The resistance can be measured in accordance to DIN EN ISO standards 16773-2 and 3, 2016.

As electrolyte a 0.9% NaCl solution is used and the impedance is measured at a temperature of 37° C. In further, in modification of DIN EN ISO 16773, the electrolyte is circulated. Hence, the impedance is measured in an environment similar to in-vivo conditions.

In particular, it has been found out that the circulation of the electrolyte results in a more reliable determination of the impedance. A distortion of the results due to adhering substances of the electrolyte on the surface of the implant is avoided.

According to a further aspect of the present disclosure, the color of the coating can be used as a marker for the corrosion resistance.

According to the present disclosure, the coating has a brightness L* in the L*a*b* color space of more than 95, preferably more than 97, in particular of 97-99.

The color seems to be also characteristic for a layer with high energy bonds.

Preferably, the coating is white. In contrary, gray coatings seem to have decreased corrosion properties.

The implant might be embodied as a granular bone filler material, as a bone plate, a screw, a nail or as an anchor.

According to an example of the present disclosure, the implant comprises a magnesium alloy, which comprises calcium, zinc and/or manganese.

Preferably, the magnesium alloy comprises at least 95% magnesium by weight.

The present disclosure further relates to a Method for determining a degradation time of a biodegradable implant, in particular of a coated Mg alloy implant, comprising the steps:
providing a bioreactor;
placing samples in the bioreactor with a salt solution;
quantifying degradation times of the samples;
feeding a database with the degradation times of the samples;
calculating a determined degradation time of the bioresorbable implant based on the degradation times of the samples.

The corrosion rate of the samples which is entered into the database can be determined by any method possible, e.g. x-ray, cutting etc.

Preferably, the corrosion rate is recorded at least with reference to the impedance.

According to a refinement of the present disclosure, the corrosion rate can be recorded with reference to other features, e.g. the alloy composition of the sample, a minimum and/or maximum diameter etc.

Preferably, the corrosion rate is calculated on basis of the electrochemical impedance spectroscopy (EIS) impedance of the implant.

Accordingly, the method enables, based on a data set, which is derived empirically, a reliable non-destructive determination of the corrosion rate of an implant.

Preferably, a temperature, a pH-value, a flow rate and/or a CO₂ value inside said bioreactor is controlled during said samples are placed in the bioreactor.

Also the determination of the impedance is preferably performed in said bioreactor. In particular, a flow of the electrolyte results in a more reliable determination of the impedance.

A flow of the electrolyte results in removing bubbles and/or concentrated salt regions from the surface. Hence, the accuracy of the impedance determination is increased.

According to an example of the present disclosure, the pH-value is controlled by introducing CO₂ into the salt solution.

It might be also suitable to apply a mechanical tension is to the sample in the bioreactor.

The present disclosure further relates to a bioreactor, being embodied to be used for a method as described above.

In particular, by using the bioreactor, the flow rate, temperature, pH-value and/or CO₂ content of the electrolyte can be controlled when measuring in the impedance of an implant.

In further, the present disclosure relates to the use of above describes method wherein a computer comprises a database which had been derived by the method according and which is used to calculate a corrosion rate of an implant based on the impedance of the implant.

In further, the present disclosure relates to a computer or data carrier comprising a program being executable to perform the calculation of the corrosion rate.

The present disclosure could be applied for different alloys metallic alloys once a hydroxyapatite is covered using a high energetic bond between the atoms.

With the present disclosure, an in-vivo degradation can be calculated.

Hence, tailor-made implants can be produced based on a calculated necessary in-vivo degradation. The shape of the implant and the thickness of the coating can be adapted to necessary demands for the lifetime of a specific implant. Due to the calculation, it is not necessary to perform a corrosion test for the implant.

The present disclosure would be able to use the method of determining a degradation time of a bioresorbable implant to perform an in-vitro-in-vivo prediction controlling the degradation rate based on the surface bonding and medical requirements.

For such, the present disclosure could create a surface which will control the degradation from the slowest to the faster.

The present disclosure will correlate and contain all the in-vitro corrosion measurements together with the in-vivo imaging data as MicroCT-CT-X-RAY and other visual imaging techniques once such will provide a degradation prediction data base.

On basis of this database, a computer program, in particular an APP on a mobile device can be provided as a tool for medical applications/predictions and for in-vitro-in-vivo simulator for biodegradable implants.

The present disclosure also refers to the use of a method of determining a degradation time of a bioresorbable implant on magnesium based alloys, most preferable above 95% wt % magnesium, without a coating.

The present disclosure could be applied for all type of medical implants including dental and veterinarian.

### BRIEF DESCRIPTION OF THE DRAWINGS

FIG. 1a is a schematic illustration of an example of a bioreactor in accordance with the principles of the present disclosure.

FIG. 1b is a schematic illustration of an example of a bioreactor in accordance with the principles of the present disclosure.

FIG. 1c is a schematic illustration of an example of a bioreactor in accordance with the principles of the present disclosure.

FIG. 2 is a flow chart showing the adjustment of a pH-value by introducing either CO₂ or water with a hydroxide in accordance with the principles of the present disclosure.

FIG. 3 is a diagram of an example of electrochemical impedance spectroscopy (EIS) equipment settings in accordance with the principles of the present disclosure.

FIG. 4 is a diagram of an example of an anodic polarization curve of a magnesium sample in accordance with the principles of the present disclosure.

FIG. 5 is a Nyquist plot of the impedance of an implant in accordance with the principles of the present disclosure.

FIG. 6 is a diagram of an example implant embodied as a bone plate in accordance with the principles of the present disclosure.

FIG. 7 is a diagram of an example implant embodied as a granular material in accordance with the principles of the present disclosure.

FIG. 8 is a diagram of a bonding energy between two atoms in accordance with the principles of the present disclosure.

FIG. 9 is a flowchart diagram of an illustrative method to produce a customized coated implant in accordance with the principles of the present disclosure.

### DETAILED DESCRIPTION

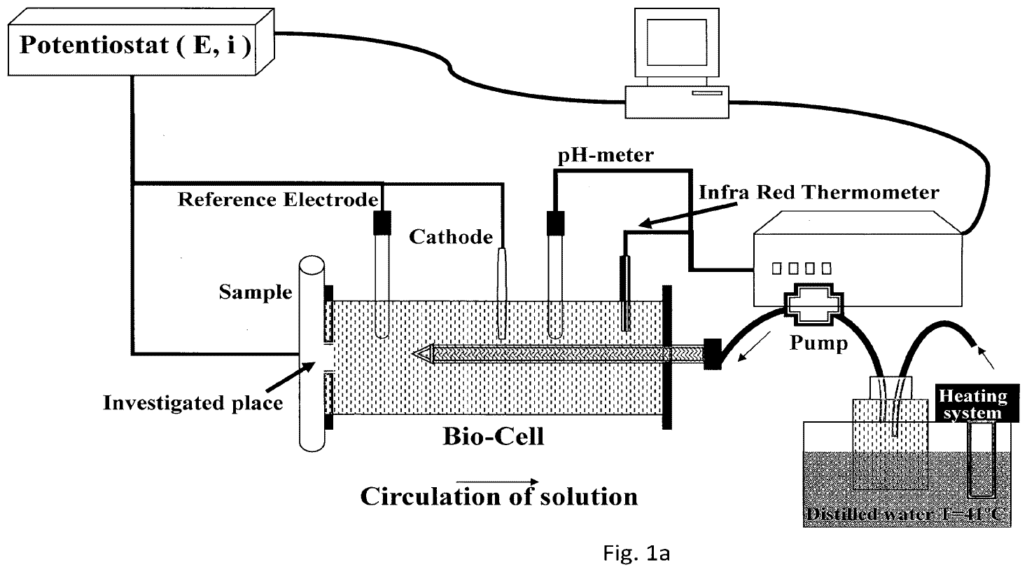

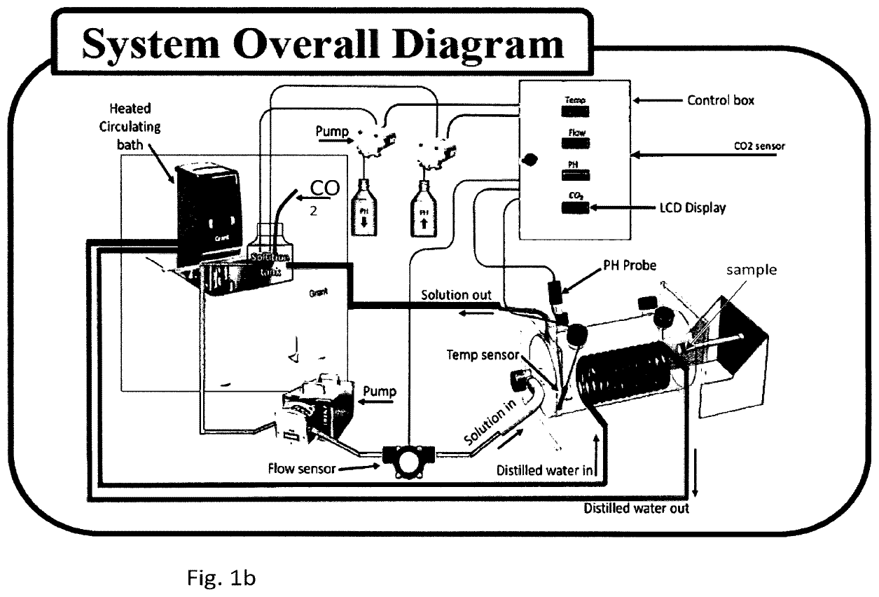

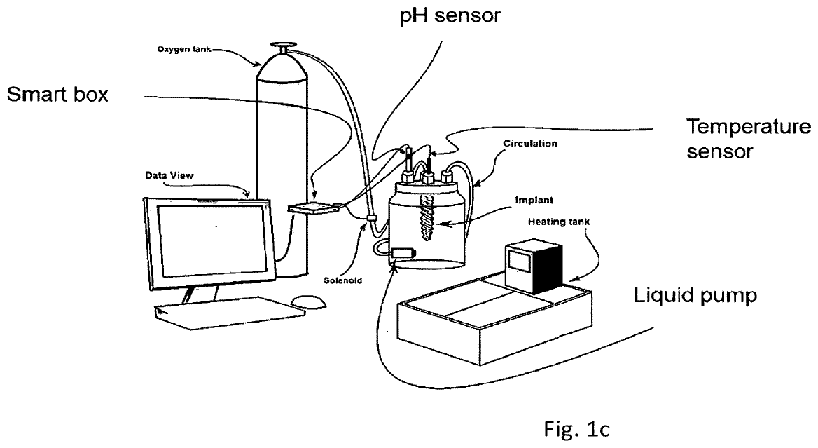

The present disclosure will be described in more detail with reference to FIG. 1 to FIG. 9.

The EIS is measured in accordance to DIN EN ISO 16773. Instead of using a standard vessel for the electrolyte, a bioreactor is used.

FIG. 1a is a schematic illustration of an example of such a bioreactor.

The bioreactor enables the simulation of in-vivo conditions and comprises a heating system, a pump, a pH-meter, a thermometer and a potentiostat.

FIG. 1b is a further schematic illustration of a bioreactor.

The system comprises a control box for regulation temperature, flow rate, pH-value and/or CO₂ content.

The control system is connected with a thermometer, a pump for infusing CO₂ and or oxygen and a CO₂ sensor.

In further, the computer is connected with a potentiostat to measure the salt concentration of the electrolyte.

The sample is introduced from the side into the bioreactor. In order to control the temperature in the bioreactor, a heating coil is connected is connected to a heating system. A pump transports water from the heating system to the heating coil inside the bioreactor.

The CO₂ is infused inside a separate vessel. This increase the accuracy.

The control box is also connected with a pump which submerged into the bioreactor. This pump circulates the water inside the bioreactor. A slow circulation is sufficient to clean the surface of the sample and to avoid the formation of gas bubbles.

The bioreactor is closed and the sample is introduced. The example according to FIG. 1c comprises a bioreactor, wherein the sample can be immersed into the electrolyte from the top.

A flow is generated by pump inside the vessel for circulating the electrolyte.

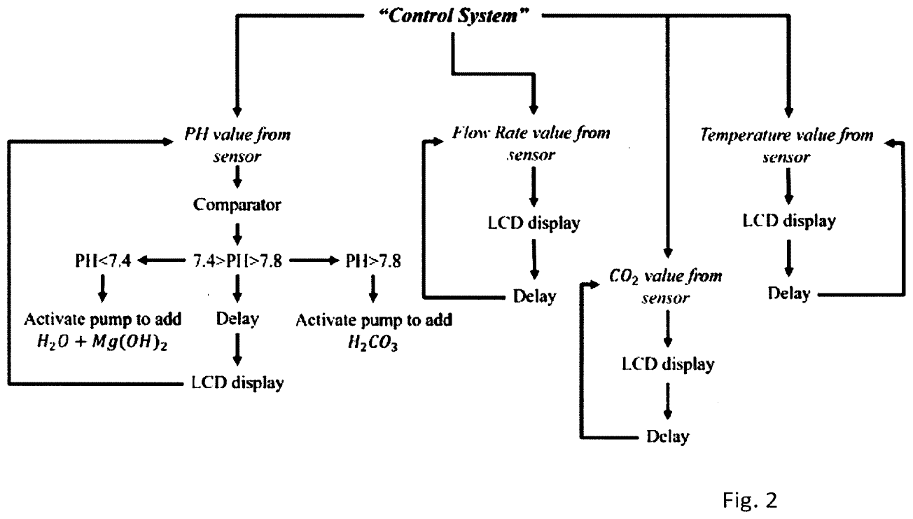

FIG. 2 is a flow chart showing that the pH-value can be adjusted by introducing either CO₂ or water with a hydroxide.

The EIS is performed at a temperature of 37° C.

For performing the EIS analysis, a Ivium-n-Stat® equipment had been used.

The EIS analysis provides a graph of Z (real impedance Zᵣₑ) versus Z′ (image impedance Zᵢₘ) usually forming a semi-circle depending on the data and the equivalent fit circuit used.

The data, if analyzed in Cartesian coordinates, is made on parameters X and Y; X being Z and Y being Z′. This is also called the Nyquist plot, which does not include the frequency which would be seen on the Bode plot. EIS helps to understand the corrosion process.

The EIS is based on an equivalent fit circuit of two resistors and one capacitor.

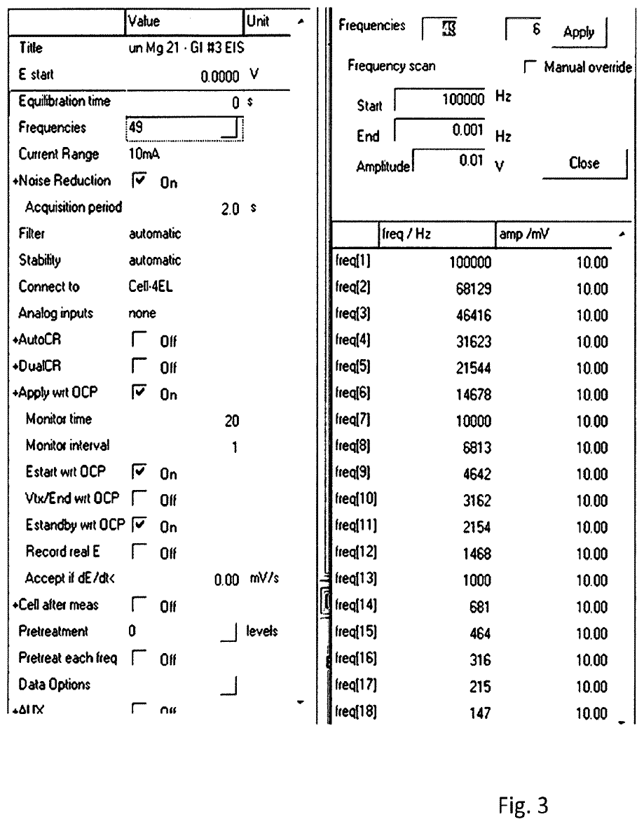

The setting of the EIS equipment is shown in FIG. 3.

The data is recorded as a function of frequency of an AC current at a fixed point. At lower frequencies and signals, that while time consuming, allow to remain in the linear potential range.

As EIS only applies small signals, it is a nondestructive technique and can be redone without ruining the surface exposed in the bio-cell.

The first frequency is usually 100 Kz and the last is 0.01 Hz, forming the desired semi-circle. The peak of the semi-circle is the value of the capacitance found experimentally. If the bounds of the circle are too big, the second frequency can be set to 0.1 Hz as well.

The reaction mechanism of Magnesium is as follows:

2 Mg₍ₛ₎+O_{2(g)}+4H⁺_{(aq)}→2Mg₂⁺_{(aq)}+2H₂O₍₁₎

Mg+2H+→Mg⁺⁺+H₂

The magnesium sample tested provides an anodic polarization curve. It begins at a low point and throughout increases in the positive potential direction. This curve provides a very important point at the “nose peak” where the sum of the cathodic and anodic rxn rates on the electrode surface is zero. The measured current is zero, which is the open circuit or rest potential point.

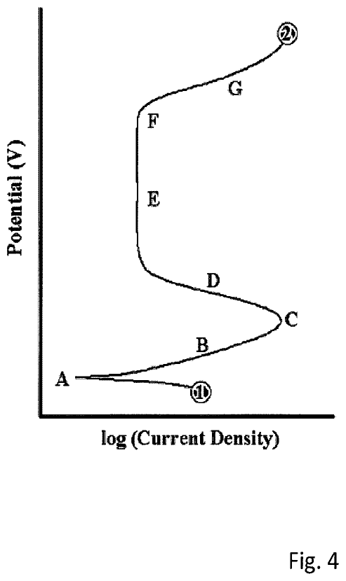

Such a curve is shown in FIG. 4, which can be interpreted as follows:
Point A Open Circuit or Rest Potential
Region B Active Region
Point C Passivation Potential
Point/Region D Current Density decreases with increasing potential
Region E Passive region
Point F Breakaway potential
Region G Applied current is rapidly increasing
Point 1 Start
Point 2 End

The samples tested were 7.75 mm circular pieces of an Mg alloy. They were prepared by being placed in a beaker with 99% alcohol, and the beaker was placed in an ultrasonic cleaner for one to five minutes.

The electrolytic solution prepared consists of a 0.9% NaCl solution, so two liters of water required 18.0 grams of NaCl. This solution was then placed over a metallic heating plate with two coated metal bars inside the beaker and left to heat and stir sufficiently.

The sample was tested in the bio corrosion cell. The NaCl water solution was distributed into two parts, one for the cell and one for the circulation bottle. A cable passes through the circulation bottle, and connects to the device that causes it to simulate circulation. This operates by simulating flow (like blood) which makes it as realistic as possible. In addition, circulation will remove magnesium corrosion products which affect the pH and replace with fresh solution without corrosion products.

On the computer, a program was used to set parameters such as temperature, pH and flow rate, they can be loaded in from pre-existing tests. The flow rate of the circulatory system is set to 100 ml/min, the pH to ˜7.4 and the temperature to approximately 37° C.±1° C. (approximate temperature of a human) depending on external factors such as temperature of the room. The heat is directly increased, decreased or set at a desired temperature via a heating bath filled with water, which the circulation bottle will be placed inside to ensure constant temperature.

A reference electrode containing calomel (Hg₂Cl₂) is placed inside the cell as a part of the circuit; in addition, there is the cathode which is made of platinum and the anode that is the sample clamped into a circular opening on the side of the cell, thus creating the circuit. The goal of the reference electrode is to be used as a point of reference in an electrochemical cell due to its stability. Ideally, the current flow through the electrode should be near zero.

The setup of the cell consists of the counter-electrode (CE), working electrode (WE) and the reference electrode (RE).

Before use of the reference electrode, it must be rinsed with distilled water to remove the crystals formed when it is idly waiting in its vial (of similar solution to the electrode)

Once the bio-cell has been set up, and connected (to the right channel), the tests can begin, but the order of the tests performed is important. The program used is IviumSoft. An Eoc (Open circuit potential) test is always performed first, followed by the EIS which is run again the following hour.

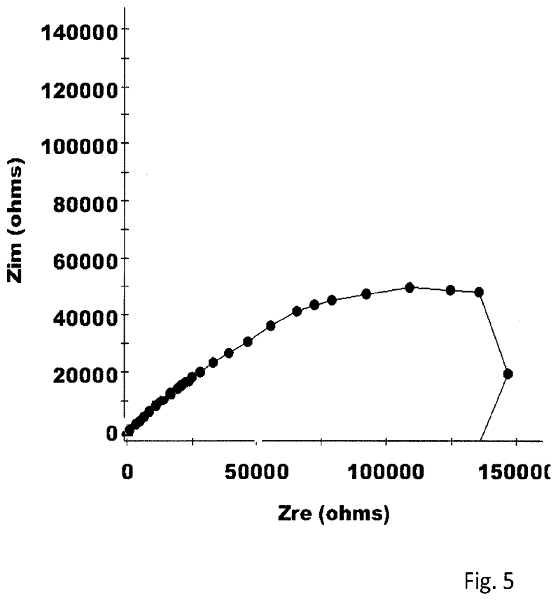

FIG. 5 is a Nyquist plot of the impedance of an implant according to the present disclosure.

Zᵣₑ reaches a value of more than 120,000Ω and Zᵢₘ a maximum value of more than 50,000Ω.

When the implant is immersed in the bioreactor, the impedance decrease due to the corrosion.

However, the impedance of the implant in the initial states can be used to calculate the corrosion rate.

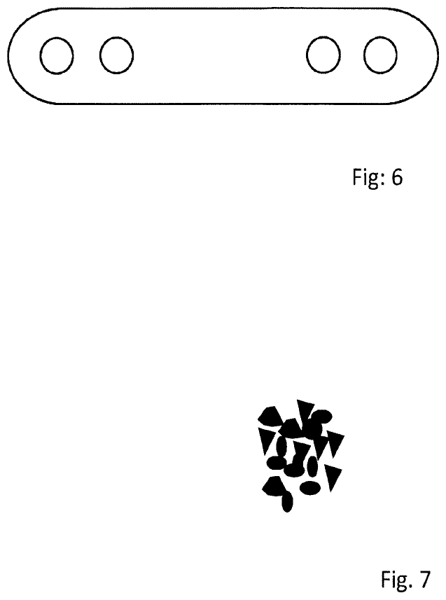

The implant can be embodied, as shown in FIG. 6 as a bone plate, comprising holes for introducing screws.

The bone plate consists of a magnesium alloy with 95 to 98% Mg and is covered with a dense hydroxyapatite layer with high energy bonds.

The hydroxyapatite layer can be applied anodic in an electrolytic bath which is a suspension comprising hydroxyapatite particles.

By using, depending on the coating device and the shape of the implant, high energy as possible for coating the implant, a white ceramic layer with a high impedance can be applied.

As illustrated in FIG. 7, the implant is, according to another example, being embodied as a granular material, comprising particles of magnesium or a magnesium alloy which are coated with hydroxyapatite.

Due to the coating, the material can be used for filling bone defects. Despite a medium particle size von less than 2 mm, the corrosion is so slow, that a formation of gas bubbles can be avoided.

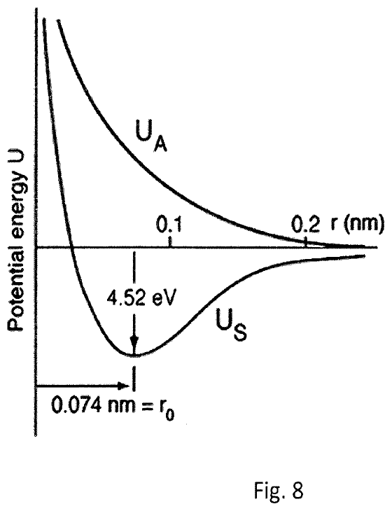

FIG. 8 is and illustration of the bonding energy between two atoms. At a specific distance, a minimum value of the potential energy is achieved.

Accordingly, this distance results in a high bond energy. It is possible to adjust the surface from lower to higher bond energy depending on the composition of the raw material alloy and the surface bonding.

A high bond energy results in a high melting temperature of the coating and a low corrosion rate.

Also, the lattice energy of the layer is a relevant factor for the corrosion resistance.

In particular, the substances of the layer have, according to an example of the present disclosure, a medium lattice energy of less than −2000 kJ/mol, in particular between −2000 and −4000 kJ/mol. The medium lattice energy can be defined as the lattice energy of the components in relation to the content of the respective component.

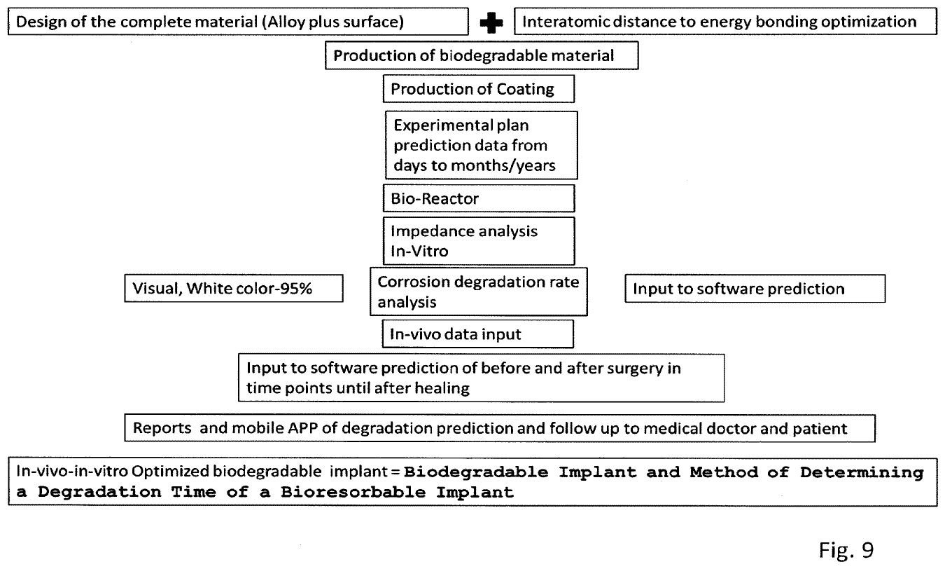

FIG. 9 is a flowchart of a method according to the present disclosure to produce a customized coated implant.

Based on an experimental plan, an in-vitro analysis of a sample can be performed by using a bioreactor.

Based on these data, a customized implant ca be produced. The in-vivo corrosion of the implant can also be used for the software, in particular for an evaluation whether the prediction of the corrosion matches with the in-vivo corrosion. This can be used to increase the accuracy of the prediction software by adapting the respective data and/or equitation.

By using the in-vitro analysis, an optimized biodegradable implant can be provided.

According to the present disclosure, a biodegradable implant with a low corrosion rate and good biocompatibility can be provided.

1. A biodegradable implant, comprising magnesium or a magnesium alloy, wherein the biodegradable implant is covered with a calcium phosphate, in particular hydroxyapatite, coating, wherein the implant has an impedance measured with electrochemical impedance spectroscopy (100 kHz-0, 1 Hz) of one or more of:
Zᵢₘ>10,000Ω
or Zᵣₑ>50,000Ω.

2. The biodegradable implant according to claim 1, wherein the coating has a brightness L* in the L*a*b* color space of more than 95.

3. The biodegradable implant according to claim 1, wherein the coating is white.

4. The biodegradable implant according to claim 1, wherein the implant includes one or more of: a granular bone filler material, a bone plate, a screw, a nail, or an anchor.

5. The biodegradable implant according to claim 1, wherein the implant comprises the magnesium alloy, and the magnesium alloy comprises one or more of: calcium, zinc or manganese, or at least 95% by weight magnesium; and
wherein the coating has an initial melting point above 1000° C.; and
wherein the coating has a medium lattice bond energy of less than −2000 kJ/mol; and
wherein the coating has an in-between medium lattice bond energy surface to a top surface with an L*a*b* color space brightness L* above 95%.

6. A method for determining a degradation time of a bioresorbable coated Mg alloy implant, comprising:
providing a bioreactor;
placing samples of the bioresorbable coated Mg alloy implant in the bioreactor with a salt solution;
quantifying degradation times of the samples;
feeding a database with the degradation times of the samples;
calculating a determined degradation time of the bioresorbable implant based on the degradation times of the samples.

7. The method according to claim 6, wherein the corrosion rate is calculated based on an EIS impedance of the implant.

8. The method according to claim 6, further comprising controlling one or more of a temperature, a pH-value, a flow rate, oxygen content, or a CO₂ value inside the bioreactor while the samples are placed in the bioreactor.

9. The method according to claim 8, wherein controlling the pH-value comprises introducing CO₂ into the salt solution.

10. The method according to claim 6, wherein the calculated determined degradation time is based on in-vivo degradation.

11. The method according to claim 6, further comprising:
producing the implant based on a calculated necessary in-vivo degradation.

12. The method according to claim 6, further comprising: storing the database; and
using the database to calculate a corrosion rate of a second implant based on an impedance of the second implant.

13. (canceled)

14. A bioreactor configured to be used for a method according to any of the preceding claims.

15. A magnesium based metal alloy which contains a coating having a lattice bond energy of less than −2000 kJ/mol and a top surface brightness L* in the L*a*b* color space of more than 95.

16. The biodegradable implant according to claim 1, wherein the calcium phosphate comprises hydroxyapatite.

17. The biodegradable implant according to claim 1, wherein the impedance measured with the electrochemical impedance spectroscopy is in the range of 25,000Ω≤Zᵢₘ≤60,000Ω and 250,000Ω≤Zᵣₑ≤300,000Ω.

18. The biodegradable implant according to claim 2, wherein brightness L* is in a range of 97-100.

19. The biodegradable implant according to claim 5, wherein the initial melting point is between 2000° and 3000° Celsius.

20. The biodegradable implant according to claim 5, wherein the lattice bond energy is between 1000 and 4000 kJ/mol.
